(19) United States
(12) Patent Application Publication
(10) Pub. No.: US 2002/0037317 A1
(43) Pub. Date: Mar. 28, 2002

(54) Wax bolus

(76) Inventor: William Leslie Porter, Diss (GB)

Correspondence Address: William M. Lee, Jr., Lee, Mann,Smith, McWilliams, Sweeney & Ohlson, 209 South LaSalle Street, Suite 410, Chicago, IL, 60604-1202, US

(21) Appl. No.: 09/962,699

(22) Filed: Sep. 25, 2001

(30) Foreign Application Priority Data
Sep. 28, 2000 (GB) 0023743.8

(51) Int. Cl. A61K 9/20; A61K 9/14

(52) U.S. Cl. 424/464; 424/484

(57) ABSTRACT

A wax bolus for ruminant animals having its melting point controlled so that the bolus is solid and stable at room temperatures but surface soft when present in the reticulo-rumen, whereby in use a beneficial substance is released from the bolus as the surface sticky bolus rubs against and repetitively attaches to and detaches itself from the stomach lining and rumen contents.

### FIELD OF THE INVENTION

This invention relates to a sustained release bolus for delivery of a beneficial substance to ruminant animals and also to a method of production of said bolus.

### BACKGROUND TO THE INVENTION

Ruminant animals, particularly when grazing, may require administration of certain substances, beneficial nutritionally or medicinally, for instance micronutrients or anthelmintics. Due to the difficulty and inconvenience of feeding supplements and administering beneficial substances to grazing animals, particularly under extensive hill or range conditions, the practice is commonly employed of administering beneficial substances in a physical form, such as boluses or rods or the like, generally referred to herein as boluses, which are retained in the ruminant fore-stomachs for prolonged periods to release contained beneficial substances from this site.

Means are known to administer, via slow release intra-ruminal boluses, water soluble beneficial substances in solid form, which may be incorporated in boluses from which they are released by leaching or by erosion.

An aim of the present invention is to provide a bolus from which the beneficial substance is released in a unique way, which enables boluses to incorporate any one of a wide variety of substances, some of which may not be conveniently released either by conventional leaching or by conventional eroding boluses.

### THE INVENTION

According to one aspect of the invention, there is provided a bolus for delivery of a substance beneficial nutritionally or medicinally to a ruminant animal, the bolus being solid and stable at room temperatures but surface-soft when present in the rumen, whereby in use the surface-soft bolus, when coming into contact with the lining of the reticulo-rumen or with solid material present in the reticulo-rumen is dissipated from the soft surface by rubbing away or by repetitive adherence to and detachment from said lining or solid contents. This action, which differs from conventional erosion, is for simplicity referred to herein as pseudo-abrasion.

Typically the bolus will incorporate a binder serving as a carrier for the beneficial substance. This binder may conveniently be a non-toxic wax or similar substance or a mixture of such substances having a melting point such that the bolus is solid and stable at room temperature but surface-soft when present in the rumen.

According to another aspect of the invention, therefore, there is provided a bolus comprising a binder, itself comprising a non-toxic wax carrying a beneficial substance in admixture therewith, the wax either in itself or in combination with another substance having a melting point such that the bolus is solid and stable at room temperature but surface soft when present in the rumen. Thus, the wax may be mixed with a melting point adjuster, usually a melting point reducer, which renders the bolus solid and stable at room temperatures but surface-soft when present in the rumen.

Two components of the bolus may typically be a wax and an oily substance as a melting point reducer. The oily substance may or may not also constitute the beneficial substance to be released. In addition the bolus will usually include a ballast material such as zinc, manganese, iron, copper powder or a mixture thereof, or copper oxide powder or particles, in order to raise the density of the bolus to a level necessary to ensure long life retention in the reticulo-rumen. Alternatively other means of retaining the bolus in the rumen may be employed, such as toggles, whereby two or more bolus bodies are connected together, or wings, whereby protruberances extend form the bolus body to minimise risk of regurgitation.

According to still another aspect of the invention, there is provided a bolus comprising a wax binder, a beneficial substance incorporated in the wax, and means for assisting retention of the bolus in the reticulo-rumen of a ruminant animal, wherein due to its melting point, the bolus is solid and stable at room temperatures but surface soft when heated in the reticulo-rumen to an extent such that the soft surface is dissipated by pseudo-abrasion.

The means of retention will typically be a densifier incorporated in the wax.

Typically, in use the bolus will be pseudo-abraded starting from one end region. It may be advantageous, since the eventually small other end region stands some risk of being passed on or regurgitated, to omit the active ingredient (beneficial substance) from that other end region, when the bolus is manufactured.

According to another aspect of the invention, the components of the bolus, including a wax, are mixed and heated to cause the wax to melt, and the mix is then cooled to form a bolus which is stable solid and non-sticky at room temperature, but surface-soft when present in the rumen.

One suitable wax is Carnauba wax.

Preferably the heated mixture, including the wax and beneficial substance, and with or without densifier, is formed into a bolus by filling into a mould and cooling to form a solid bolus. Alternatively, the mix may be formed with particles, powder, granules or blocks, before or after solidification, and the solid material may be formed into a bolus, conveniently a cylinder with at least one rounded end, by compression or, after reheating, by injection moulding.

Where the beneficial substance constitutes the oily substance incorporated in the bolus as melting point reducer, the oily substance may consist, for example, of vitamin A, vitamin D3 or vitamin E. Alternatively the beneficial oily substance may comprise a bloat control oil, such as Soya bean oil, a nutritionally beneficial fatty acid such as essential fatty acids or non-essential fats and oils such as lecithins, or a combination of one or more of these substances.

However, it is alternatively possible to include an oily substance simply for the purpose of adjusting the overall melting point of the finished bolus, the beneficial substance being any one or more of a wide variety of substances which have been found to be nutritionally or medicinally beneficial to ruminant animals either for general health reasons, to promote growth, or to prevent diseases which can occur in ruminant animals.

A preferred wax or wax mixture, which is employed as a carrier and binder for the beneficial substance, is a wax or wax mixture which is non-toxic and miscible with the oily substance or substances. Such a wax or wax mixture preferably has a melting point of from 45 to 110 degrees centigrade, more especially 50 to 100 degrees centigrade, preferably 60 to 90 degrees centigrade, even more preferably 70 to 88 degrees centigrade and ideally from 75 to 85 degrees centigrade.

Additionally, the technique of the present invention may be employed to deliver solid beneficial substances into the reticulo-rumen, in the form of powders, dust, granules or particles which, in manufacture, may be mixed with the molten wax, together with or without an oily beneficial substance or substances. In this case, the solid beneficial substances are released progressively from the bolus as the surface-soft wax pseudo-abrades.

Examples of solid beneficial substances which may be delivered in this way are salts of trace elements, eg sodium selenite, calcium iodate, cobalt sulphate, copper sulphate, vitamins such as biotin or folic acid, antibiotics, hormones, growth promoters and anthelmintics.

As is made clear in the foregoing, even if the oily substance is the beneficial substance, other beneficial substances may also be included in the bolus, including the aforementioned solid beneficial substances.

### DESCRIPTION OF EMBODIMENT

An example of bolus in accordance with the invention was prepared as follows: 3 g of Carnauba wax was melted at 110° C. 5 g of dlα tocopheryl acetate was added which was stirred together with 45 g of zinc powder, average particle size 120 microns. The mixture was filled into a two part, cylindrical longitudinally divided mould and allowed to cool to room temperature. The mould was then separated and the resultant bolus was removed.

The bolus produced according to the above described method was firm and non-sticky to the touch at room temperature (approximately 18° C.) but when heated in an incubator to a temperature approximating the that of the reticulo-rumen (39° C.) the surface became sticky to the touch.

The bolus, weighing 53 g, was then tested by placing in the rumen of a rumen-fistulated bullock for a total of fifteen days. In that time, due to the pseudo-abrasion in the ruminant fore-stomach, it lost a total of 35.94 g, ie 67.8% of its initial weight.

Such a bolus would be expected to have a total release period of approximately twenty-two days. This could readily be prolonged by known techniques, such as total or partial coating with an abradable covering such as beeswax, or with a principally insoluble and non-abradable coating such as polyethylene.

In the above example, the beneficial substance is constituted by the oily substance, namely tocopheryl acetate. However this will not necessarily always be the case. The oily substance may simply be incorporated as a melting point reducer for the wax.

Additionally, the invention makes possible the production of a pulse release bolus, wherein predetermined doses of beneficial substance are released from the bolus at intervals of time, eg one every few days, or every few weeks. This may be achieved by providing the bolus with a brittle skin, say of thin non-toxic plastics material, having an opening through which the bolus body, surface-soft when in the rumen, is exposed at one end. The bolus is formed in layers alternately containing and not containing the beneficial substance. As the bolus body is slowly dissipated from the exposed end, the skin breaks away progressively and successively to expose the bolus layers.

1. A bolus for delivery of a nutritionally or medicinally beneficial substance to a ruminant animal, the bolus being solid and stable at room temperatures but surface-soft when present in the rumen, whereby in use the surface-soft bolus, when coming into contact with the lining of the reticulo-rumen or with solid material present in the reticulo-rumen is dissipated from the soft surface by rubbing away or by repetitive adherence to and detachment from said lining or solid contents.

2. A bolus according to claim 1, incorporating a binder serving as a carrier for the beneficial substance.

3. A bolus according to claim 2, wherein the binder is, or comprises, a non-toxic wax, said binder having a melting point such that the bolus is solid and stable at room temperature but surface-soft when present in the rumen.

4. A bolus according to claim 3, wherein the wax is mixed with a melting point adjuster, which renders the bolus solid and stable at room temperatures but surface-soft when present in the rumen.

5. A bolus according to claim 4, wherein the melting point adjuster is a melting point reducer.

6. A bolus according to claim 5, wherein the melting point reducer is an oily substance.

7. A bolus according to claim 6, wherein the oily substance constitutes the beneficial substance.

8. A bolus according to claim 1, comprising means for assisting retention of the bolus in the reticulo-rumen of a ruminant animal.

9. A bolus according to claim 8, including a ballast material in order to raise the density of the bolus to a level necessary to ensure long life retention in the reticulo-rumen.

10. A bolus according to claims 8, including a toggle for retaining them in the rumen, whereby two or more bolus bodies will connect together.

11. A bolus according to claim 8, wherein the means of retention comprises a densifier incorporated in the wax.

12. A bolus according to claim 1, wherein the beneficial substance is omitted from at least one end region of the bolus body, when the bolus is manufactured.

13. A bolus according to claim 1, comprising Carnauba wax.

14. A method of manufacture of the bolus of claim 1, wherein the components of the bolus, including a wax, are mixed and heated to cause the wax to melt, and the mix is then cooled to form a bolus which is stable solid and non-sticky at room temperature, but surface-soft when present in the rumen.

15. A method according to claim 14, wherein the wax is Carnauba wax.

16. A method according to claim 14, wherein the heated mixture, including the wax and beneficial substance, is formed into a bolus by filling into a mould and cooling to form a solid bolus.

17. A method according to claim 16, wherein the mixture includes a densifier.

18. A method according to claim 16, wherein the beneficial substance constitutes an oily substance incorporated in the bolus as a melting point reducer and said oily substance is selected from the group consisting of vitamin A, vitamin D3, vitamin E, a bloat control oil, and a nutritionally beneficial fatty acid.

19. A method according to claim 18, wherein the wax, which is employed as a carrier and binder for the beneficial substance, is non-toxic and miscible with the oily substance.

20. A method according to claim 14, wherein the wax has a melting point in the range 60 to 90 degrees centigrade.

21. A method according to claim 20, wherein the wax has a melting point in the range 70 to 88 degrees Centigrade.

22. A method according to claim 20, wherein the wax has a melting point in the range 75 to 85 degrees Centigrade.
